(19) United States
(12) Patent Application Publication
(10) Pub. No.: US 2025/0012704 A1
(43) Pub. Date: Jan. 9, 2025

(54) MICROFLUIDIC CARTRIDGE AND METHODS OF USE THEREOF

(71) Applicants: RizLab Health, Inc., Princeton, NJ (US); Rutgers, The State University of New Jersey, New Brunswick, NJ (US)

(72) Inventors: Mehdi JAVANMARD, Princeton, NJ (US); Jianye SUI, Princeton, NJ (US); Shahriar AZIZPOUR, Princeton, NJ (US); Zhongtian Lin, Princeton, NJ (US)

(73) Assignees: RizLab Health, Inc., Princeton, NJ (US); Rutgers, The State University of New Jersey, New Brunswick, NJ (US)

(21) Appl. No.: 18/705,588

(22) PCT Filed: Oct. 26, 2022
(86) PCT No.: PCT/US2022/078708

§ 371 Date: Apr. 29, 2024

Related U.S. Application Data
(60) Provisional application No. 63/272,414, filed on Oct. 27, 2021.

(51) Int. Cl. G01N 15/12; B01L 3/00; G01N 15/01; G01N 15/10

(52) U.S. Cl. CPC G01N 15/12; B01L 3/502715; G01N 15/01; B01L 2300/0645; B01L 2300/087; G01N 2015/1006; G01N 2015/135

(57) ABSTRACT

This disclosure provides a microfluidic system (e.g., microfluidic cartridge, microfluidic chip) comprising two or more microfluidic flow channels for impedance-based detection of a biological entity in a sample. The disclosed system enables simultaneous measurements of a sample in two or more microfluidic flow channels to minimize faulty results. It eliminates the need of lysing samples and measuring them multiple times that is more time-consuming, and could introduce some variations across samples and devices.

### CROSS-REFERENCE TO RELATED APPLICATIONS

This application claims priority under 35 U.S.C. § 119 (e) to U.S. Provisional Patent Application No. 63/272,414, filed Oct. 27, 2021. The foregoing application is incorporated by reference herein in its entirety.

### STATEMENT REGARDING FEDERALLY SPONSORED RESEARCH

This invention was made with government support under Grant Number 2025773 from National Science Foundation, Contract Number 75A50119C00048 from the Department of Health and Human Services, Office of the Assistant Secretary for Preparedness and Response, and Biomedical Advanced Research and Development Authority, DRIVe, and Grant Number NNX16AO69A from the Translational Research Institute through NASA. The government has certain rights in the invention.

### FIELD

This disclosure relates to microfluidic flow systems for impedance-based detection of a biological entity in a sample and methods of use thereof.

### BACKGROUND

Generally, flow cytometry is a specialized technology whereby cells, biomarkers, and particles are quantified. Cell counting is an application of flow cytometry and can provide significant insight into a patient's health. A well-known example includes a complete blood count (CBC) test, which can yield information about low or high red blood cell (RBC), white blood cell (WBC), or platelet levels amongst many other specific biomarker counts.

Current approaches for counting cells commonly employ fluorescence or impedance-based measurements. Fluorescence-based cytometers require labeling of biological cells with antibodies functionalized with fluorophores. Continuous cell counting has been demonstrated in vivo using fluorescence-based flow cytometers. Impedance cytometry, which utilizes electrical measurements, is an alternative technique that does not require a labeling procedure. Impedance cytometry can be used to detect cells, proteins, and nucleic acids.

However, impedance cytometry requires expensive data acquisition hardware to read highly sensitive signals that are often buried in background noise. Furthermore, the baseline of the signal may drift over an extended period, reducing the amount of post-gain amplification that can be applied to the signal. Moreover, it becomes more challenging when multiple measurements of a sample are needed, because such multiple measurements are not only time-consuming but also prone to introducing variations across samples and measuring devices.

Accordingly, there remains a need for new and improved systems and methods for impedance measurements.

### SUMMARY

This disclosure addresses the need mentioned above in a number of aspects. In one aspect, this disclosure provides a microfluidic system (e.g., microfluidic cartridge, microfluidic chip) for impedance-based detection of a biological entity in a sample. The system comprises: (a) a substrate; (b) two or more microfluidic flow channels positioned on the substrate, wherein the microfluidic flow channels are configured to conduct passage of the biological entity; (c) at least one inlet formed on the substrate, wherein the at least one inlet is configured to receive the sample and in fluid communication with the microfluidic flow channels; (d) at least one outlet formed on the substrate, wherein the at least one outlet is in fluid communication with the microfluidic flow channels and configured to receive the sample after the sample flows through the microfluidic channels; and (e) an impedance circuit disposed on the substrate, comprising two or more excitation electrodes and a common electrode, wherein each of the excitation electrodes is respectively coupled to each of the microfluidic flow channels and configured to be electrically connected to a signal generator, wherein the excitation electrodes are configured to receive and electrically communicate an excitation signal applied by the signal generator to each of the microfluidic flow channels, wherein the excitation signal generates an electric field between each of the excitation electrodes and the common electrode, and wherein the common electrode is coupled to all of the microfluidic flow channels and configured to be electrically connected to an impedance analyzer, wherein the common electrode is configured to electrically communicate an output signal to the impedance analyzer, and wherein the output signal correlates to an impedance variation caused by displacement of the biological entity within each of the microfluidic flow channels.

In some embodiments, the microfluidic flow channels are formed on or affixed to the substrate. In some embodiments, the microfluidic flow channels are configured to conduct passage of the biological entity therethrough simultaneously. In some embodiments, the microfluidic flow channels comprise three microfluidic flow channels.

In some embodiments, the microfluidic flow channels are of the same dimension. In some embodiments, the microfluidic flow channels comprise a microfluidic flow channel having a width of from about 70 to about 90 micrometers and a height of from about 18 to about 22 micrometers. In some embodiments, the microfluidic flow channel has a width of about 80 micrometers and a height of about 20 micrometers. In some embodiments, the microfluidic flow channels have a circular, oval, or polygonal cross-section.

In some embodiments, the at least one inlet comprises three inlets, and the at least one outlet comprises three outlets. In some embodiments, the at least one inlet comprises one inlet, and the at least one outlet comprises three outlets.

In some embodiments, the excitation electrodes or the common electrode have a width of from about 10 to about 50 micrometers. In some embodiments, the excitation electrodes or the common electrode have a width of about 25 micrometers. In some embodiments, the excitation electrodes are spatially disposed on the substrate with a gap between two electrodes of from about 10 to about 50 micrometers. In some embodiments, the gap between two electrodes is about 20 micrometers.

In some embodiments, the inlet or the outlet has a diameter of from about 2 to about 8 centimeters. In some embodiments, the inlet has a diameter of about 3 centimeters, and the outlet has a diameter of about 5 centimeters.

In some embodiments, the signal generator comprises a function generator. In some embodiments, the impedance analyzer comprises a lock-in amplifier.

In some embodiments, the output signal is proportional to the impedance variation of the biological entity within the each of the microfluidic flow channels. In some embodiments, the excitation signal has a frequency of from about 100 kHz to about 20 MHz. In some embodiments, the signal generator applies a different frequency of the excitation signal to each of the excitation electrodes.

In some embodiments, the microfluidic flow channels comprise three microfluidic flow channels, and the signal generator applies three different frequencies of the excitation signal respectively to the three microfluidic channels. In some embodiments, the three different frequencies are about 490 kHz, about 500 kHz, and about 510 kHz, respectively. In some embodiments, the excitation signal comprises sinusoidal excitation signals.

In some embodiments, the impedance analyzer demodulates impedance responses of the microfluidic flow channels from the output signal received from the common electrode.

In some embodiments, the substrate is formed of a polymer material. In some embodiments, the substrate is formed of polymethyl methacrylate (PMMA) or fluorine-doped tin oxide (FTO)/PMMA.

In some embodiments, the system comprises two layers of the substrate, wherein the two layers of the substrate are patterned with metal and affixed to each other by adhesive, wherein space generated by the adhesive forms the microfluidic flow channels. In some embodiments, the adhesive comprises pressure sensitive adhesive.

In some embodiments, the microfluidic flow channels of a size of about 25 micrometers.

In some embodiments, the two layers of the substrate comprise a glass layer. In some embodiments, the two layers of the substrate are patterned by laser patterning. In some embodiments, the metal comprises indium tin oxide, fluorine tin oxide, gold, aluminum, platinum, graphene, graphene oxide, reduced graphene oxide, molebdium disulfide, silver, silver chloride, copper, graphite, titanium, steel, brass, or a combination thereof.

In some embodiments, the biological entity comprises a bacterium, a virus, a protein, a microparticle, a nanoparticle, a nucleic acid, a biomarker, or a bead with a biological material attached thereto.

In some embodiments, the biological entity comprises any one of red blood cell, white blood cell, platelet, hematocrit, hemoglobin, neutrophil, lymphocyte, microbial, and a combination thereof.

In another aspect, this disclosure provides a kit comprising a microfluidic system as described herein.

In another aspect, this disclosure provides a method for identifying or counting a biological entity in a sample. The method comprises: (i) providing the microfluidic system as described herein; (ii) applying the sample to the at least one inlet; (iii) applying an excitation signal to the excitation electrodes by the signal generator for a period of time; (iv) receiving an output signal communicated from the common electrode; (v) determining an impedance variation caused by displacement of the biological entity within the microfluidic flow channels; and (vi) determining a type or a number of the biological entity in the sample based on the impedance variation.

In another aspect, this disclosure also provides a method of diagnosing a disease or disorder in a subject. The method comprises: (a) providing the microfluidic system as described herein; (b) applying the sample to the at least one inlet; (c) applying an excitation signal to the excitation electrodes by the signal generator for a period of time; (d) receiving an output signal communicated from the common electrode; (e) determining an impedance variation caused by displacement of the biological entity within the microfluidic flow channels; (f) determining a number of the biological entity in the sample based on the impedance variation; and (g) determining that the subject has the disease or disorder if a difference between the number of the biological entity and a control level is greater than a threshold value.

In another aspect, this disclosure further provides a method of monitoring progression of a disease or disorder in a subject. The method comprises: (i) providing the microfluidic system as described herein; (ii) applying the sample to the at least one inlet; (iii) applying an excitation signal to the excitation electrodes by the signal generator for a period of time; (iv) receiving an output signal communicated from the common electrode; (v) determining an impedance variation caused by displacement of the biological entity within the microfluidic flow channels; (vi) determining a number of the biological entity in the sample based on the impedance variation and determining if the number of the biological entity is elevated or decreased as compared to a second control level; and (vii) determining that (a) the subject has progression of the disease or disorder if the number of the biological entity is elevated as compared to the second control level; and (b) the subject has regression of the disease or disorder if the number of the biological entity is decreased as compared to the second control level.

In some embodiments, the excitation signal has a frequency of from about 100 kHz to about 20 MHz. In some embodiments, the method comprises applying by the signal generator a different frequency of the excitation signal to each of the excitation electrodes. In some embodiments, the microfluidic flow channels comprise three microfluidic flow channels; and the method comprises applying by the signal generator three different frequencies of the excitation signal respectively to the three microfluidic channels. In some embodiments, the three different frequencies are about 490 kHz, about 500 kHz, and about 510 kHz, respectively. In some embodiments, the excitation signal comprises sinusoidal excitation signals.

In some embodiments, the method further comprises demodulating by the impedance analyzer impedance responses of the microfluidic flow channels from the output signal received from the common electrode. In some embodiments, the method comprises applying a wavelet filter to the output signal. In some embodiments, the method comprises applying a Hampel filter to the output signal.

In some embodiments, the biological entity comprises a bacterium, a virus, a protein, a microparticle, a nanoparticle, a nucleic acid, a biomarker, or a bead with a biological material attached thereto. In some embodiments, the biological entity comprises any one of red blood cell, white blood cell, platelet, hematocrit, hemoglobin, neutrophil, lymphocyte, microbial, and a combination thereof.

In some embodiments, the method comprises determining the number of one or more of white blood cells, lymphocytes, and neutrophils in the sample. In some embodiments, the method comprises determining a neutrophil:lymphocyte ratio. In some embodiments, the method comprises identifying a disease or disorder or monitoring progression of the disease or disorder by comparing the neutrophil:lymphocyte ratio to a control ratio.

In some embodiments, the method comprises identifying a disease or disorder or monitoring progression of the disease or disorder based on one or more characteristics selected from white blood cell counts, concentration of neutrophils, percentage of neutrophils, volume of neutrophils, concentration of lymphocytes, percentage of lymphocytes, volume of neutrophils, volume of lymphocytes, and neutrophil to lymphocyte ratio. In some embodiments, identifying a disease or disorder or monitoring progression of the disease or disorder is performed by a machine learning module.

In some embodiments, the disease or disorder is a bacterial or viral infection. In some embodiments, the disease or disorder comprises influenza or SARS-COV-2.

In some embodiments, the sample comprises a bodily fluid. In some embodiments, the bodily fluid comprises blood. In some embodiments, the method further comprises contacting the sample with a lysis reagent for a period of time. In some embodiments, the method further comprises quenching the sample after the lysis step.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

The foregoing will be apparent from the following more particular description of example embodiments, as illustrated in the accompanying drawings in which reference characters refer to the same parts throughout the different views. The drawings are not necessarily to scale, emphasis instead being placed upon illustrating embodiments.

FIGS. 1A and 1B are a set of diagrams showing examples of microfluidic cartridges. FIG. 1A shows a schematic diagram of an example microfluidic cartridge. FIG. 1B shows an image of an example microfluidic cartridge.

FIG. 2 shows an example signal processing strategy. A wavelet filter was applied to reduce the noise and interference in the raw signal. A Hampel filter was implemented to remove the baseline drift in the signal.

FIG. 3 shows an example peak classification strategy.

FIGS. 4A, 4B, and 4C are a set of graphs showing the correlation analysis between Cytotracker results and Beckman Coulter hematology analyzer results on WBC concentration (FIG. 4A), neutrophil concentration (FIG. 4B), and lymphocyte concentration (FIG. 4C). Thirty blood samples were tested.

FIGS. 5A, 5B, 5C, and 5D are a set of diagrams showing an example FTO/PMMA cartridge (FIG. 5A), FTO coated glass (FIG. 5B), and double adhesive tape (FIG. 5C). FIG. 5D shows the current response of FTO/PMMA cartridge when several polystyrene beads flowing through the sensing region.

FIG. 6 is a diagram showing an example microfluidic cartridge with a second layer inlet to increase the flow rate of a sample in the microfluidic channels.

FIG. 7 is a diagram showing an example package for connecting to a microfluidic cartridge as disclosed.

FIGS. 8A and 8B are a set of diagrams showing multi-parametric analysis to identify a disease or disorder. FIG. 8A shows multi-parametric analysis that provides a unique signature for a disease or disorder. FIG. 8B shows constellation diagrams (image classification algorithms).

FIG. 9 shows a example study workflow of a machine learning-based analysis for correlation analysis to diagnose infections in patients.

FIGS. 10A, 10B, 10C, and 10D show the results of a correlation analysis comparing measurements by a cytotrakcer and a Horiba device.

FIG. 11 shows the results of a single dimension analysis comparing measurements by a Beckman Coulter and a cytotracker.

FIG. 12 shows a machine learning matrix and AUC from measurements by a Beckman Coulter and a cytotracker.

FIG. 13 shows an example microfluidic cartridge formed from two metal-patterned glass layers affixed with adhesive.

FIG. 14 shows an example microfluidic system as provided as a test strip that can be used as a plug-n-play device.

### DETAILED DESCRIPTION

This disclosure provides a microfluidic system (e.g., microfluidic cartridge, microfluidic chip) comprising two or more microfluidic flow channels for impedance-based detection of a biological entity in a sample. The disclosed system enables simultaneous measurements (e.g., doublet, triplet, quadruplet) of a sample in two or more microfluidic flow channels to minimize faulty results. It eliminates the need of lysing samples and measuring them multiple times that is more time-consuming and could introduce some variations across samples and devices. In addition, the use of averaging of the concentration calculated over the multitude of channels results in better accuracy of the blood cell measurement.

### Microfluidic Systems

Accordingly, in one aspect, this disclosure provides a microfluidic system (e.g., microfluidic cartridge, microfluidic chip) for impedance-based detection of a biological entity in a sample. The system comprises: (a) a substrate; (b) two or more (e.g., 2, 3, 4, 5, 6, 7, 8, 9, 10) microfluidic flow channels positioned on the substrate, wherein the microfluidic flow channels are configured to conduct passage of the biological entity; (c) at least one inlet formed on the substrate, wherein the at least one inlet is configured to receive the sample and in fluid communication with the microfluidic flow channels; (d) at least one outlet formed on the substrate, wherein the at least one outlet is in fluid communication with the microfluidic flow channels and configured to receive the sample after the sample flows through the microfluidic channels; and (e) an impedance circuit disposed on the substrate, comprising two or more excitation electrodes and a common electrode, wherein each of the excitation electrodes is respectively coupled to each of the microfluidic flow channels and configured to be electrically connected to a signal generator, wherein the excitation electrodes are configured to receive and electrically communicate an excitation signal applied by the signal generator to each of the microfluidic flow channels, wherein the excitation signal generates an electric field between each of the excitation electrodes and the common electrode, and wherein the common electrode is coupled to all of the microfluidic flow channels and configured to be electrically connected to an impedance analyzer, wherein the common electrode is configured to electrically communicate an output signal to the impedance analyzer, and wherein the output signal correlates to an impedance variation caused by displacement of the biological entity within each of the microfluidic flow channels.

In some embodiments, the microfluidic flow channels are formed on or affixed to the substrate. In some embodiments, the microfluidic flow channels are configured to conduct passage of the biological entity therethrough simultaneously. In some embodiments, the microfluidic flow channels comprise three microfluidic flow channels.

In some embodiments, the microfluidic flow channels are of the same dimension. In some embodiments, the microfluidic flow channels comprise a microfluidic flow channel having a width of from about 70 to about 90 micrometers and a height of from about 18 to about 22 micrometers. In some embodiments, the microfluidic flow channel has a width of about 80 micrometers and a height of about 20 micrometers. In some embodiments, the microfluidic flow channels have a circular, oval, or polygonal cross-section.

In some embodiments, the at least one inlet comprises three inlets, and the at least one outlet comprises three outlets. In some embodiments, the at least one inlet comprises one inlet, and the at least one outlet comprises three outlets.

In some embodiments, the excitation electrodes or the common electrode have a width of from about 10 to about 50 micrometers. In some embodiments, the excitation electrodes or the common electrode have a width of about 25 micrometers. In some embodiments, the excitation electrodes are spatially disposed on the substrate with a gap between two electrodes of from about 10 to about 50 micrometers. In some embodiments, the gap between two electrodes is about 20 micrometers.

In some embodiments, the inlet or the outlet has a diameter of from about 2 to about 8 centimeters. In some embodiments, the inlet has a diameter of about 3 centimeters, and the outlet has a diameter of about 5 centimeters.

In some embodiments, the signal generator comprises a function generator (e.g., a two-channel function generator). In some embodiments, each electrode pair is electrically connected to an impedance analyzer. In some embodiments, the impedance analyzer comprises a lock-in amplifier.

The impedance can be analyzed or measured in any suitable frequency range, e.g., a frequency range between about 1 Hz and about 100 MHz, or between 10 Hz and about 5 MHz. In some embodiments, the excitation signal has a frequency of from about 100 kHz to about 20 MHz. In some embodiments, the signal generator applies a different frequency of the excitation signal to each of the excitation electrodes.

In some embodiments, the microfluidic flow channels comprise three microfluidic flow channels, and the signal generator applies three different frequencies of the excitation signal respectively to the three microfluidic channels. In some embodiments, the three different frequencies are about 490 kHz, about 500 kHz, and about 510 kHz, respectively. In some embodiments, the excitation signal comprises sinusoidal excitation signals.

In some embodiments, the output signal is proportional to the impedance variation of the biological entity within the each of the microfluidic flow channels. In some embodiments, the impedance analyzer demodulates impedance responses of the microfluidic flow channels from the output signal received from the common electrode.

After the data is sampled and converted to digital, the signal undergoes denoising and also detrending (to remove any drift present in the solution). Multiple different signal processing algorithms can be employed, including wavelets, band-pass filters, and also low-pass filters for denoising. For detrending, median filters, high-pass filters, band-pass filters, and also wavelet filters may be used. Peaks may then then identified using a thresholding function. If multiple peaks exist together, the two are decoupled from each other.

The term “microfluidic system,” or “microfluidic flow system,” as used herein, refers to a fluidic system including one or more channels in the micrometer range (which may also be referred to as microchannels) where a sample volume may be provided to flow in and along the microchannels based on fluidic motion. In some embodiments, the microfluidic system may be formed on a microchip to form a microfluidic chip.

The term “microfluidic chip,” as used herein, refers to a chip having at least one microfluidic channel having a cross-sectional area of less than 1 mm² and a length of at least 1 mm. In some embodiments, the microfluidic chip has a plurality of microfluidic channels, for example, at least 2, 3, 4, 5, 6, 7, 8, 9, or 10 microfluidic channels. In some embodiments, the microfluidic chip has at least one microfluidic channel having a length of at least 1 mm (e.g., 2 mm, 4 mm, 6 mm, 8 mm, 10 mm). In some embodiments, the microfluidic chip comprises a plurality of layers, for example, at least 2, 3, 4, 5, 6, 7, 8, 9, or 10 layers.

In some embodiments, a microfluidic channel may have a non-polygonal cross-section, for example, a circular, oval, or other non-polygonal cross-section. In some embodiments, a microfluidic channel may have a polygonal cross-section, for example, a triangular, rectangular, or other polygonal cross-section.

The term “electrical impedance,” “impedance,” or “cell impedance,” as used herein, generally refers to a measure of the difficulty an electrical current faces when it traverses through a biological entity. Electrical impedance can be the ratio of the voltage to the current, and given in the units of Ohms. Electrical impedance can be measured by applying a known voltage and measuring the electrical current or by applying a known electrical current and measuring the resulting voltage. In either case, a direct current (DC) or preferably an alternating current (AC) can be used. The AC waveform can be in the form of a sinusoidal current, a square wave, a pulse train, or any other repeating form.

The term “impedance changes” or “impedance variation,” as used herein, refers to changes in impedance detected at the detection electrode. The changes may include changes in amplitude, phase, or amplitude and phase of the signal.

The term “in fluid communication” in relation to the different sections of a microfluidic system refers to a communication between two sections of the microfluidic system. In some embodiments, this communication may be a direct connection or a direct path between two sections of the microfluidic system or may include one or more intervening sections in the path between two sections of the microfluidic system.

The term “in electrical communication with the microfluidic channel” as applied to the electrodes means that the electrodes are in direct contact with the fluids analyzed in the microfluidic channel. The term “Electronic communication,” as used herein, refers to the connection between the electrical elements of the system, either directly or wirelessly.

The term “biological entity,” as used herein, refers to a biomarker, a cell, an organelle, a virus particle, a biopolymer, or a combination thereof. The term “cell” may include a eukaryotic cell or a prokaryotic cell. The term “cell” may also include a peripheral blood mononuclear cell, a cell of the immune system including a white blood cell, a T cell and a T helper cell, a biomarker including a circulating tumor cell, a lymphocyte, a CD4 lymphocyte, and an endothelial progenitor cell. The term “eukaryotic cell” may include a mammalian cell or a yeast cell. The term “mammalian cell” may include a tumor cell, a blood cell, a cell of the immune system, a progenitor cell, and a fetal cell. The term “biopolymer” may include a polypeptide, a nucleic acid, a lipid, and an oligosaccharide. In some embodiments, the biological entity may have a DNA anchor for incubation and capture on the surface of the microelectrode array.

As used herein, “sample” refers to anything which may contain a moiety to be isolated, manipulated, measured, quantified, detected, or analyzed using the disclosed microfluidic flow systems or methods. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid. Biological tissues are aggregates of cells, usually of a particular kind together with their intercellular substance that form one of the structural materials of a human, animal, plant, bacterial, fungal, or viral structure, including connective, epithelium, muscle, and nerve tissues. Examples of biological tissues also include organs, tumors, lymph nodes, arteries, and individual cell(s). The biological samples may further include cell suspensions, solutions containing biological molecules (e.g., proteins, enzymes, nucleic acids, carbohydrates, chemical molecules binding to biological molecules).

As used herein, a “bodily fluid sample” or “fluid sample,” or the like in the context of obtaining a sample from a patient, subject or individual refers to a sample which may be blood plasma, blood serum, whole blood, CSF, urine, saliva, tears, semen, colostrum or any recoverable bodily fluid as obtained from the individual for C-TM testing in one or more of the various assays disclosed herein.

In some embodiments, the biological entity comprises a bacterium, a virus, a protein, a microparticle, a nanoparticle, a nucleic acid, a biomarker, or a bead with a biological material attached thereto. In some embodiments, the biological entity comprises any one of red blood cell, white blood cell, platelet, hematocrit, hemoglobin, neutrophil, lymphocyte, microbial, and a combination thereof.

An “electrode,” as used herein, is a structure having a high conductivity, that is, a conductivity much higher than the surrounding material. As used herein, an “electrode structure” refers to a single electrode, particularly one with a complex structure (as, for example, a spiral electrode structure), or a collection of at least two electrode elements that are electrically connected together. All the electrode elements within an “electrode or “one or structure” are electrically connected. Non-limiting examples of materials for electrodes or electrode elements are indium tin oxide (ITO), chromium, gold, copper, nickel, platinum, silver, steel, and aluminum. Electrodes can comprise more than one material. Choice of appropriate materials for making electrodes depends on several factors: whether the material is conductive enough, how difficult it is for patterning such material on a substrate, whether the material can be reliably used for performing molecular detection assay of the present invention.

In some embodiments, the substrate is formed of a polymer material. Examples of such polymers include, without limitation, poly-etheretherketones (PEEK), polyetherketones (PEK), polyphenylene sulfides (PPS), polyethylene sulfide (PES), polyetherimides (PEI), polyvinylidene fluoride (PVDF), polysulfones (PSU), polycarbonates (PC), polyphenylene ethers, aromatic thermoplastic poly-esters, aromatic polysulfones, thermoplastic polyimides, liquid crystal polymers, thermoplastic elastomers, polyethylene, polypropylene, polystyrene (PS), acrylics, such as polymethyl-methacrylate (PMMA), polyacrylonitrile (PAN), acrylonitrile butadiene styrene (ABS), and the like, ultra-high-molecular-weight polyethylene (UHMWPE), polytetrafluoroethylene (PTFE/Teflon®), polyamides (PA) such as nylons, polyphenylene oxide (PPO), polyoxymethylene plastic (POM/Acetal), polyarylether-ketones, polyvinylchloride (PVC), mixtures thereof and the like.

In some embodiments, the substrate is formed of polymethyl methacrylate (PMMA) or fluorine-doped tin oxide (FTO)/PMMA.

In some embodiments, the system comprises two layers of the substrate. In some embodiments, the two layers of the substrate are patterned with metal and affixed to each other by adhesive. In some embodiments, space generated by the adhesive forms the microfluidic flow channels. In some embodiments, the adhesive comprises pressure sensitive adhesive.

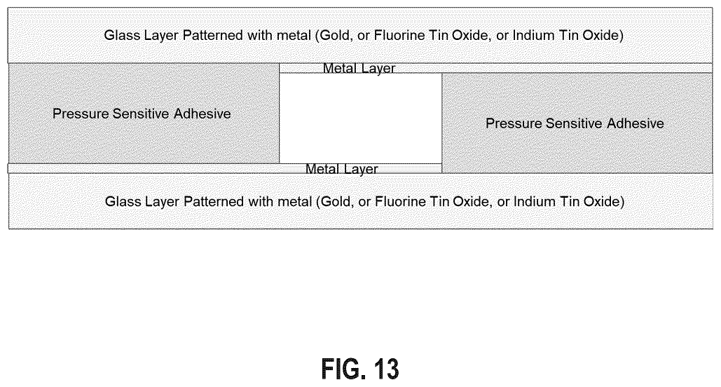

FIG. 13 shows an example microfluidic cartridge formed from two metal-patterned glass layers affixed with adhesive. In this configuration, electrodes can be manufactured using laser patterning on two layers of substrates. The two glass layers can be affixed with pressure sensitive adhesive between them. The adhesive may be about 25 micrometers thick, which serves as the channel layer. The adhesive also results in bonding to the top glass/electrode layer and bottom glass/electrode layer. Using this configuration, laser-based patterning techniques can be used, which is very cheap and effective for manufacturing, and also create a distance between electrodes as small as 25 micrometers, necessary to achieve high sensitivity.

In some embodiments, the microfluidic flow channels of a size of from about 10 to about 50 micrometers (e.g., about 25 micrometers).

In some embodiments, the two layers of the substrate comprise a glass layer. In some embodiments, the two layers of the substrate are patterned by laser patterning. In some embodiments, the metal comprises indium tin oxide, fluorine tin oxide, gold, aluminum, platinum, graphene, graphene oxide, reduced graphene oxide, molebdium disulfide, silver, silver chloride, copper, graphite, titanium, steel, or brass.

In some embodiments, the microfluidic flow channels can be removably mounted to or formed on the substrate. Similarly, a microfluidic cartridge can be removably mounted to a support or package. In some embodiments, the support or package may include ports connected to, e.g., in electrical communication with, a signal generator and/or a signal analyzer. For example, the microfluidic cartridge can be configured as a plug-and-play cartridge suitable for receiving and analyzing different types of biological entities. In one example, the microfluidic flow channel can be configured for receiving red blood cells (RBCs), white blood cells (WBCs), hematocrit, hemoglobin, or a combination thereof. In another example, the microfluidic flow channel can be configured for receiving neutrophils, lymphocytes, or a combination thereof. In another example, the microfluidic flow channel can be configured for receiving microbial cells. In yet another example, the microfluidic flow channel can be configured for receiving and analyzing proteins present in blood or saliva, i.e., blood or saliva proteomics analysis.

In another aspect, this disclosure provides a kit comprising a microfluidic system as described herein. In some embodiments, the kit may optionally include an apparatus for collecting a sample (e.g., bodily fluid). In some embodiments, the apparatus for collecting a sample may include, without limitation, a capillary tube, a pipette, a syringe, a needle, a pump, and a swab. In some embodiments, the kit may include informational material, e.g., instruction material. The informational material can be descriptive, instructional, marketing, or other material that relates to the microfluidic flow systems described herein.

### Methods of Use

In another aspect, this disclosure provides a method for identifying or counting a biological entity in a sample. The method comprises: (i) providing the microfluidic system as described herein; (ii) applying the sample to the at least one inlet; (iii) applying an excitation signal to the excitation electrodes by the signal generator for a period of time; (iv) receiving an output signal communicated from the common electrode; (v) determining an impedance variation caused by displacement of the biological entity within the microfluidic flow channels; and (vi) determining a type or a number of the biological entity in the sample based on the impedance variation.

In another aspect, this disclosure also provides a method of diagnosing a disease or disorder in a subject. The method comprises: (a) providing the microfluidic system as described herein; (b) applying the sample to the at least one inlet; (c) applying an excitation signal to the excitation electrodes by the signal generator for a period of time; (d) receiving an output signal communicated from the common electrode; (e) determining an impedance variation caused by displacement of the biological entity within the microfluidic flow channels; (f) determining a number of the biological entity in the sample based on the impedance variation; and (g) determining that the subject has the disease or disorder if a difference between the number of the biological entity and a control level is greater than a threshold value.

In another aspect, this disclosure further provides a method of monitoring progression of a disease or disorder in a subject. The method comprises: (i) providing the microfluidic system as described herein; (ii) applying the sample to the at least one inlet; (iii) applying an excitation signal to the excitation electrodes by the signal generator for a period of time; (iv) receiving an output signal communicated from the common electrode; (v) determining an impedance variation caused by displacement of the biological entity within the microfluidic flow channels; (vi) determining a number of the biological entity in the sample based on the impedance variation and determining if the number of the biological entity is elevated or decreased as compared to a second control level; and (vii) determining that (a) the subject has progression of the disease or disorder if the number of the biological entity is elevated as compared to the second control level; and (b) the subject has regression of the disease or disorder if the number of the biological entity is decreased as compared to the second control level.

Also within the scope of this disclosure is a method of diagnosing a disease or disorder in a subject based on a machine learning classifier. Unlike many existing methods in the art, the disclosed method may include performing a multivaraite analysis on types and concentrations of a variety of biology particles and using one or more machine learning classifiers to determine the disease or disorder in the subject.

The terms “patient,” “individual,” and “subject” are used interchangeably and generally refer to any living organism to which the disclosed methodology is utilized to obtain a bodily fluid sample in order to perform a diagnostic or monitoring method described herein. A patient can be an animal, such as a human. A patient may also be a domesticated animal or a farm animal. A “patient” or “individual” may also be referred to as a subject.

As used herein, a “control” level refers, in some embodiments, to a level of a biological entity in a sample obtained from one or more individuals who do not suffer from a disease or disorder that is of interest in the investigation. The level may be measured on an individual-by-individual basis or on an aggregate basis, such as an average. A “control” level can also be determined by analysis of a population of individuals who have the disease or disorder but are not experiencing an acute phase of the disease or disorder. In some embodiments, a “control” level of a biological entity in a sample is obtained from the same individual for whom a diagnosis is sought or whose condition is being monitored, but is obtained at a different time. In some embodiments, a “control” level of a biological entity in a sample can refer to a level of a biological entity in a sample obtained from the same patient at an earlier time, e.g., weeks, months, or years earlier.

As used herein, “the determined level is elevated as compared to the control level” refers to a positive change in value from the control level. As used herein, “the determined level is decreased as compared to the control level” refers to a negative change in value from the control level.

In some embodiments, the excitation signal has a frequency of from about 100 kHz to about 20 MHz. In some embodiments, the method comprises applying by the signal generator a different frequency of the excitation signal to each of the excitation electrodes. In some embodiments, the microfluidic flow channels comprise three microfluidic flow channels; and the method comprises applying by the signal generator three different frequencies of the excitation signal respectively to the three microfluidic channels. In some embodiments, the three different frequencies are about 490 kHz, about 500 kHz, and about 510 kHz, respectively. In some embodiments, the excitation signal comprises sinusoidal excitation signals.

In some embodiments, the differences between the frequencies of the two adjacent microfluidic flow channels (e.g., |f1-f2|, |f2-f3|, wherein f1, f2, and f3 are the frequencies of three microfluidic flow channels) can be the same or different. In some embodiments, the differences between the frequencies of the two adjacent microfluidic flow channels can be a 2% to 10% increment of the lower frequency. Non-limiting example frequency sets include 490 kHz, about 500 kHz, and about 510 kHz; about 0.9 MHz, about 1 MHZ, and about 1.1 MHz; and about 9 MHZ, about 10 MHz, and about 1 MHz.

In some embodiments, the method further comprises demodulating by the impedance analyzer impedance responses of the microfluidic flow channels from the output signal received from the common electrode. In some embodiments, the method comprises applying a wavelet filter to the output signal. In some embodiments, the method comprises applying a Hampel filter to the output signal.

In some embodiments, the biological entity comprises a bacterium, a virus, a protein, a microparticle, a nanoparticle, a nucleic acid, a biomarker, or a bead with a biological material attached thereto. In some embodiments, the biological entity comprises any one of red blood cell, white blood cell, platelet, hematocrit, hemoglobin, neutrophil, lymphocyte, microbial, and a combination thereof.

In some embodiments, the method comprises determining the number of one or more of white blood cells, lymphocytes, and neutrophils in the sample. In some embodiments, the method comprises determining a neutrophil:lymphocyte ratio. In some embodiments, the method comprises identifying a disease or disorder or monitoring progression of the disease or disorder by comparing the neutrophil:lymphocyte ratio to a control ratio.

In some embodiments, the method comprises identifying a disease or disorder or monitoring progression of the disease or disorder based on one or more characteristics selected from white blood cell counts, concentration of neutrophils, percentage of neutrophils, volume of neutrophils, concentration of lymphocytes, percentage of lymphocytes, volume of neutrophils, volume of lymphocytes, and neutrophil to lymphocyte ratio. In some embodiments, identifying a disease or disorder or monitoring progression of the disease or disorder is performed by a machine learning module.

After the data is sampled and converted to digital, the signal undergoes denoising and also detrending (to remove any drift present in the solution). Multiple different signal processing algorithms can be employed, including wavelets, band-pass filters, and also low-pass filters for denoising. For detrending, median filters, high-pass filters, band-pass filters, and also wavelet filters may be used. Peaks may then then identified using a thresholding function. If multiple peaks exist together, the two are decoupled from each other. Machine learning algorithms such as support vector machine, neural networks, may also been tested for classification of the peaks. Peak data obtained can be analyzed in a fully automated manner using a combination of signal processing and also artificial intelligence. Both supervised and unsupervised learning classifiers can be used. Different cell types such as platelets, RBCs, and WBCs can be classified based on impedance peak response. Even white blood cells can be differentiated from each other (e.g., lymphocytes, neutrophils, monocytes, etc.). It is also possible to classify cells expressing certain antigens (e.g., CD4 positive and CD4 negative).

In addition, an impedance cytometer incorporating the disclosed microfluidic cartridge may be trained by running pure samples of each cell type through the microfluidic cartridge. Features such as peak amplitude at different frequencies, peak area, half-width maximum, cepstral intensity, etc., can be used to improve accuracy. In addition to this, machine learning can be used to correlate disease states to blood cell counts. For example, based on the percentage of lymphocytes with respect to total white blood cell count or percentage of neutrophil with respect to total white blood cell count, infections can be classified as viral (increase in lymphocyte percentage) or bacterial (increase in neutrophil). Neutrophil and lymphocyte cell population data (cell volume and conductivity) can also provide more specificity regarding if an infection is viral or bacterial. The combination of the impedance cytometry data and the symptoms can be used as a feature for training a machine learning classifier to accurately classify disease states.

In some embodiments, the disease or disorder is a bacterial or viral infection. In some embodiments, the disease or disorder comprises influenza or SARS-COV-2.

In some embodiments, the sample comprises a bodily fluid. In some embodiments, the bodily fluid comprises blood. In some embodiments, the method further comprises contacting the sample with a lysis reagent for a period of time. In some embodiments, the method further comprises quenching the sample after the lysis step.

In some embodiments, the microfluidic flow channel may be configured to receive a biological entity suspended in a bodily fluid (e.g., blood) or a buffer solution.

### Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

As used herein, the terms “subject,” “patient,” or “living being” are used interchangeably irrespective of whether the subject has or is currently undergoing any form of treatment. As used herein, the terms “subject” and “subjects” may refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgus monkey, chimpanzee, etc.) and a human). The subject may be a human or a non-human. In this context, a “normal,” “control,” or “reference” subject, patient, or population is/are one(s) that exhibit(s) no detectable disease or disorder, respectively.

“Sample,” “test sample,” and “patient sample” may be used interchangeably herein. The sample can be a sample of, serum, urine plasma, amniotic fluid, cerebrospinal fluid, cells (e.g., antibody-producing cells) or tissue. Such a sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. The terms “sample” and “biological sample” as used herein generally refer to a biological material being tested for and/or suspected of containing an analyte of interest such as antibodies. The sample may be any tissue sample from the subject. The sample may comprise protein from the subject.

Any cell type, tissue, or bodily fluid may be utilized to obtain a sample. Such cell types, tissues, and fluid may include sections of tissues such as biopsy and autopsy samples, frozen sections 20) taken for histologic purposes, blood (such as whole blood), plasma, serum, sputum, stool, tears, mucus, saliva, hair, skin, red blood cells, platelets, interstitial fluid, ocular lens fluid, cerebral spinal fluid, sweat, nasal fluid, synovial fluid, menses, amniotic fluid, semen, etc. Cell types and tissues may also include lymph fluid, ascetic fluid, gynecological fluid, urine, peritoneal fluid, cerebrospinal fluid, a fluid collected by vaginal rinsing, or a fluid collected by vaginal flushing. A tissue or cell type May be provided by removing a sample of cells from an animal, but can also be accomplished by using previously isolated cells (e.g., isolated by another person, at another time, and/or for another purpose). Archival tissues, such as those having treatment or outcome history, may also be used. Protein purification may not be necessary.

Methods well known in the art for collecting, handling, and processing urine, blood, serum, and plasma, and other body fluids, can be used in the practice of the present disclosure, for instance, when the antibodies provided herein are employed as immunodiagnostic reagents, and/or in an immunoassay kit. The test sample can comprise further moieties in addition to the analyte of interest, such as antibodies, antigens, haptens, hormones, drugs, enzymes, receptors, proteins, peptides, polypeptides, oligonucleotides or polynucleotides. For example, the sample can be a whole blood sample obtained from a subject. It can be necessary or desired that a test sample, particularly whole blood, be treated prior to immunoassay as described herein, e.g., with a pretreatment reagent. Even in cases where pretreatment is not necessary, pretreatment optionally can be done for mere convenience (e.g., as part of a regimen on a commercial platform). The sample may be used directly as obtained from the subject or following a pretreatment to modify a characteristic of the sample. Pretreatment may include extraction, concentration, inactivation of interfering components, and/or the addition of reagents.

The terms “determining,” “measuring,” “assessing,” and “assaying” are used interchangeably and include both quantitative and qualitative measurement, and include determining if a characteristic, trait, or feature is present or not. Assessing may be relative or absolute. “Assessing the presence of” a target includes determining the amount of the target present, as well as determining whether it is present or absent.

As used herein, the term “diagnosis” means detecting a disease or disorder or determining the stage or degree of a disease or disorder. Usually, a diagnosis of a disease or disorder is based on the evaluation of one or more factors and/or symptoms that are indicative of the disease. That is, a diagnosis can be made based on the presence, absence or amount of a factor which is indicative of the presence or absence of the disease or condition. Each factor or symptom that is considered to be indicative of the diagnosis of a particular disease does not need to be exclusively related to the particular disease; i.e. there may be differential diagnoses that can be inferred from a diagnostic factor or symptom. Likewise, there may be instances where a factor or symptom that is indicative of a particular disease is present in an individual that does not have a particular disease. The diagnostic methods may be used independently or in combination with other diagnosing and/or staging methods known in the medical art for a particular disease or disorder.

The term “prognosis” as used herein refers to a prediction of the probable course and outcome of a clinical condition or disease. A prognosis is usually made by evaluating factors or symptoms of a disease that are indicative of a favorable or unfavorable course or outcome of the disease. The phrase “determining the prognosis” as used herein refers to the process by which the skilled artisan can predict the course or outcome of a condition in a patient. The term “prognosis” does not refer to the ability to predict the course or outcome of a condition with 100% accuracy instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

The word “substantially” does not exclude “completely,” e.g., a composition which is “substantially free” from Y may be completely free from Y. Where necessary, the word “substantially” may be omitted from the definition of the invention.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1

As disclosed herein, this example demonstrates a novel method to improve the detection accuracy and develop a metric to flag the false test. A microfluidic chip was designed to enable 3-parallel measurements at the same time. The lysed whole blood was pipetted into and measured in three separate channels. The data are collected and analyzed. The cell flow speed and the number of cells flowing through the channel were derived from the data. The cell concentration was calculated as the cell count divided by the flow rate in unit time. The average of cell concentrations calculated separately from three different measurements was used to reduce the random error to improve the accuracy. The coefficient of variation (CV) of three measurements results was calculated, which was the standard deviation divided by the average. If the CV was above 15%, the test was flagged as faulty, and the outlier was identified within three measurements based on the differences between every two values. If one measurement result was much larger or smaller than the other two results, it was eliminated, and the average of the rest two results was calculated. In this way, the outlier was removed, and the accuracy of the results was improved.

In some applications, the whole blood was lysed with a buffer containing 0.12% v/v formic acid and 0.05% w/v saponin. Then quenched by the solution containing 0.6% w/v sodium carbonate and 3% w/v sodium chloride.

In one example, the disclosed microfluidic platform, for performing three measurements on the same device simultaneously, includes three microfluidic channels and four electrodes. There are two approaches to perform the measurement, using the main electrode as excitation and monitor the branch electrodes or vice versa. The detection utilizes electrical impedance sensing technology. A 20 μl aliquot of lysed sample was pipetted into the channel inlet. Under the gravity and capillary force, the cell flows into the channel towards the outlet. When a cell flows through the sensing region, it partially blocks the AC electrical field generated between two electrodes and thus causes an increase in electrical impedance. The sensing electrode is connected to a lock-in amplifier to measure the electrical signals. The data are transferred to a PC for downstream analysis.

The device can discriminate between viral and bacterial infections. It does so by measuring the neutrophil levels, the lymphocyte levels, the total WBC count, the respective cell volumes, and the neutrophil to lymphocyte ratio. These parameters are used together and combined in a multi-parametric machine learning classifier and then assess whether the infection is viral or bacterial.

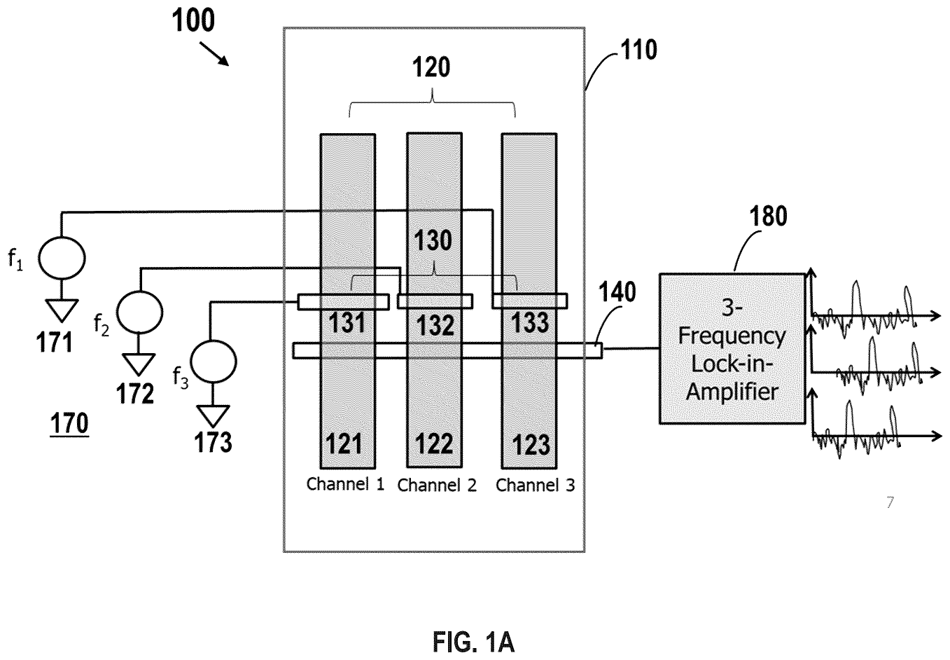

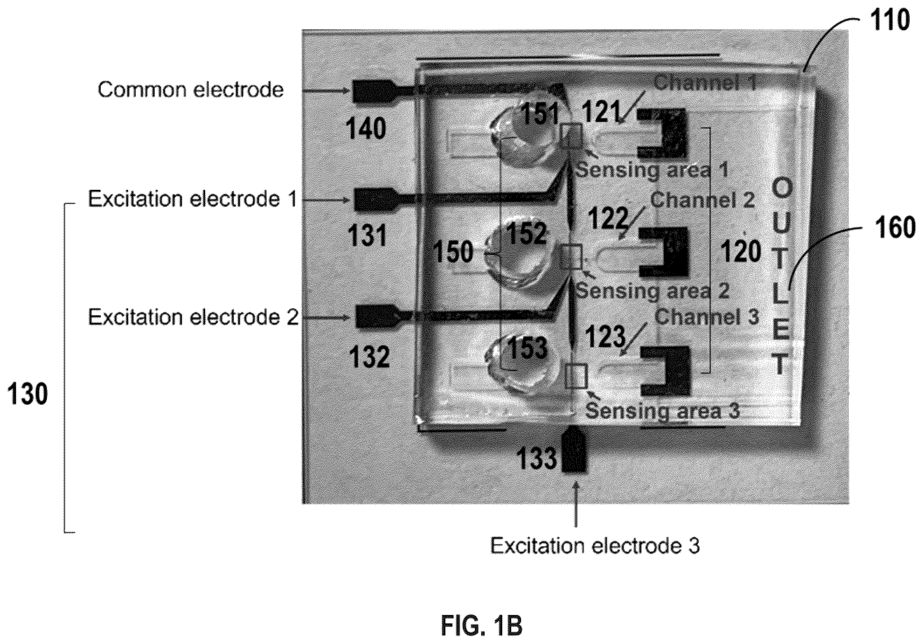

With reference to FIGS. 1A and 1B, a cartridge is provided for performing 3-parallel electrical impedance measurements at the same time. A shown in FIGS. 1A and 1B, the microfluidic cartridge 100 includes three microfluidic channels 120 (e.g., 121, 122, 123) and four electrodes (130, such as 131, 132, 133; and 140). Three different microfluidic channels (121, 122, 123) share the same outlet (150, 160). Three excitation electrodes (131, 132, 133) are tied with three different excitation sources (170, such as 171, 172, 173). The frequencies of three sinusoidal excitation signals are carefully chosen, where they are probing the same property of the blood cell while they are different enough to avoid interference from each other. The fourth electrode or the common electrode (140) is connected to a lock-in amplifier (180) (Zurich Instruments MLFI, Zurich, Switzerland) to monitor the impedance change in three channels (121, 122, 123). When a cell flows through the sensing region, it partially blocks the AC electrical field generated between two electrodes and thus causes an increase in electrical impedance. The common electrode (140) adds up the impedance responses measured in three separate channels (121, 122, 123). The response of each channel is then demodulated by the lock-in amplifier (180) at the same specific frequency used for excitation. The data are sent to a local computer and analyzed using MATLAB (MathWorks, Natick, MA, USA).

Electrodes were patterned on a 3-inch fused silica wafer using standard photolithography procedures. A thin layer of positive photoresist (AZ5214, MicroChemicals GmbH) was spin-coated on the wafer. After pre-bake, mask and wafer alignment, UV exposure, development, and post-bake, the desired pattern was transferred from the mask to the wafer. The metals, 5 nm chromium and 100 nm gold, were deposited sequentially using electron beam evaporation, whereby the chromium layer was used for enhancing the adhesion of gold film on glass. Submerging the wafer in acetone lifted off unwanted gold. The resultant electrodes were 25 μm in width, and the gap between the two electrodes was 20 μm. The SU-8 (negative photoresist) silicon mold for the microfluidic channel was fabricated using a similar process. The size of the channel is 80 μm in width and 20 μm in height. The channel pattern was transferred from the mold to a PDMS slab using the following process. PDMS polymer and curing agent (Sylgard 184, Dow Corning) at a 10:1 ratio were mixed sufficiently. Then, the mixture was poured onto the channel mold, degassed to remove bubbles in the mixture, and baked at 80° C. for 30 minutes to allow for curing. Afterwards, the PDMS channel was peeled off, and three holes were punched through the PDMS to be used as an inlet reservoir (3 mm in diameter). The microfluidic channel and the electrodes on the glass substrate were aligned and bonded.

The concentration of red blood cells (RBCs) in whole blood is 1000× higher than the concentration of white blood cells (WBCs). To accurately detect WBCs and simplify the process and analysis, RBCs were lysed before measuring the WBC concentration. The lysing solution contains 0.12% v/v formic acid and 0.05% w/v saponin. The quenching solution contains 0.6% w/v sodium carbonate and 3% w/v sodium chloride. The lysis solution has a low pH, which is around 2.6-4.0, and a low osmolarity, less than 50 mOsm, and is selective toward RBC since leukocytes are more resistant than erythrocytes in hypotonic solutions. The formic acid is the main component that enables RBC lysis; the saponin reduces the debris volume. The quench solution has a high pH, around 10, so that the pH of the lysed blood sample is attained within the range 7-7.5, and high osmolarity, around 1000 mOsm, in order to prevent WBC damage and to preserve the chemical balance of leukocytes. 10 μl whole blood was lysed with 120 μl lysing solution with continual agitation to enhance mixing. Then, 53 μl of quenching solution was added to the mixture to halt the lysis reaction. Three 20 μl aliquots of RBC lysed product was pipetted into the inlet of every channel. The fluid in the channel was driven by a combination of capillary force and the pressure gradient induced by the fluid height difference between inlet and outlet. The impedance across the two electrodes was changed when a cell flowed through the sensing region because the electric field was blocked by the cell. The impedance changes in three channels were captured by a lock-in amplifier. The data were demodulated, sent to a local computer for downstream analysis. The data were collected for five minutes. To extract impedance data from the raw impedance flow cytometry signals, a customized signal processing algorithm was employed.

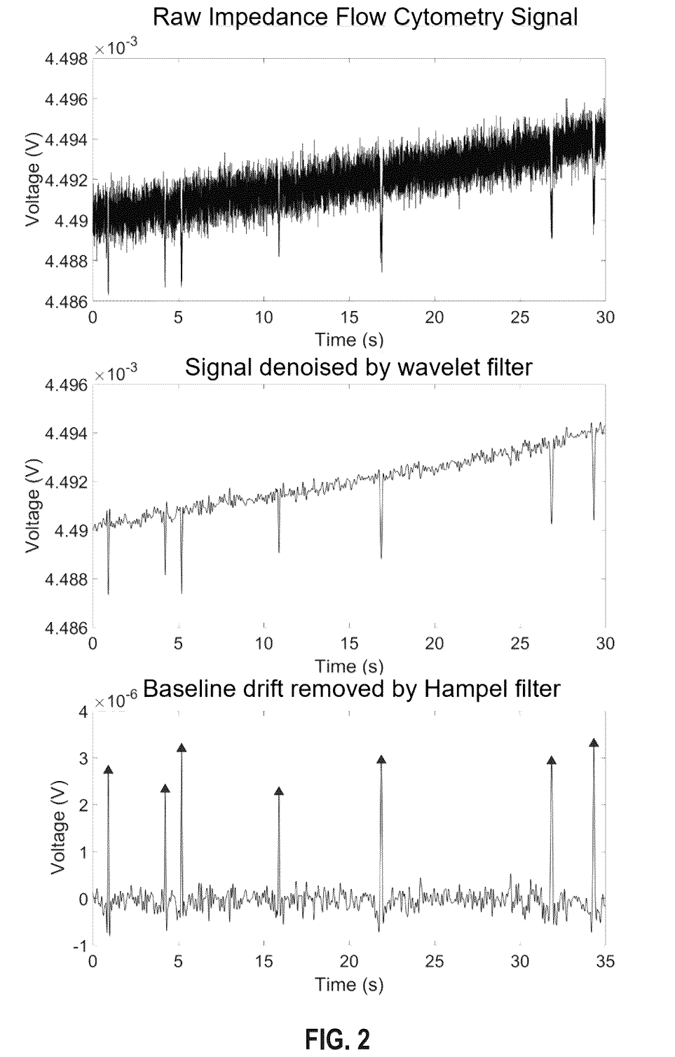

As shown in FIG. 2, the raw signal includes thermal noise, flicker noise, and shot noise. Additionally, the temperature fluctuation in the environment, the complex electrochemical interactions between electrodes and electrolyte, and the non-uniformity of electrical properties of electrolyte in the channel due to the flow also induced noises and baseline drift to cytometry signals. To reduce the noise and remove the baseline drift in raw signals, a wavelet filter and Hampel filter were applied sequentially in signal processing. Wavelet analysis takes advantage of the windowing technique with a variable size window and produces information in both the time domain and frequency domain. The SNR increased from around 2 to around 6 after removing the noise, as depicted in FIG. 2. To eliminate the baseline drift in the signal sufficiently, a smooth baseline was identified by implementing the Hampel filter. The Hampel filter replaces the value that lies far from the median in the data window with the median value. The signal got flipped after detrending and had an almost flat baseline at 0. Then the impedance peak was identified by setting an amplitude threshold.

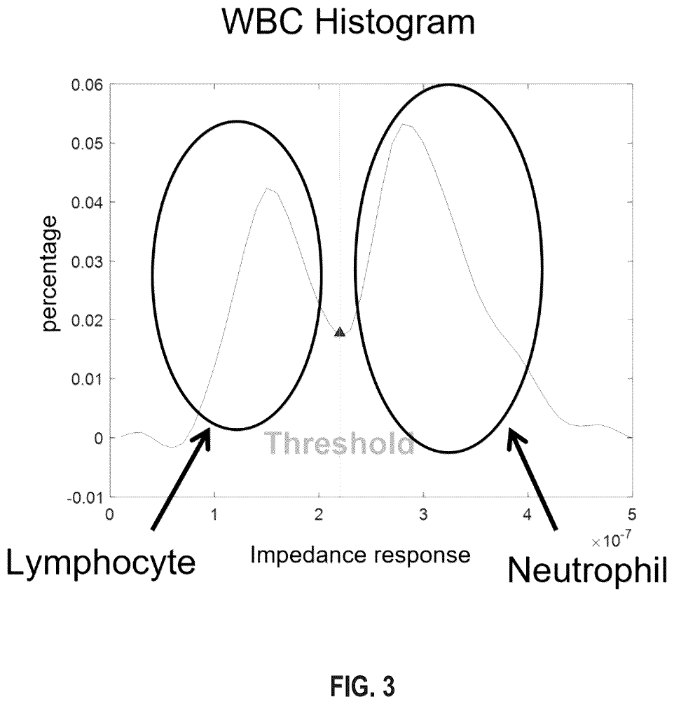

The cell count was obtained from impedance cytometry data. The peak width was identified, which is the transit time of the cells flowing through the electrodes, to determine fluid velocity. Given the fluid velocity and the volume across the electrodes, the flow rate was calculated. Then, by dividing the cell count in unit time by the flow rate, the WBC concentration in the lysed sample can be derived. The neutrophils and lymphocytes were classified based on the impedance peak amplitude distribution, as shown in FIG. 3. The peak amplitude histogram of WBC was plotted and smoothened with a wavelet filter at level 2. The threshold of lymphocyte and neutrophil was determined by the corresponding peak amplitude of the local minima. Following a similar procedure described previously, lymphocyte concentration and neutrophil concentration were calculated. Cell concentrations calculated separately from three different measurements were utilized to reduce the random error to improve the accuracy. The coefficient of variation (CV) of three measurements results was calculated, which was the standard deviation divided by the average. If the CV was above 15%, the test was flagged as faulty, and the outlier within three measurements was identified based on the differences between every two values. If one measurement result was much larger or smaller than the other two results, it was eliminated, and the average of the rest two results was calculated. In this way, the accuracy of the results was improved after the outlier was removed.

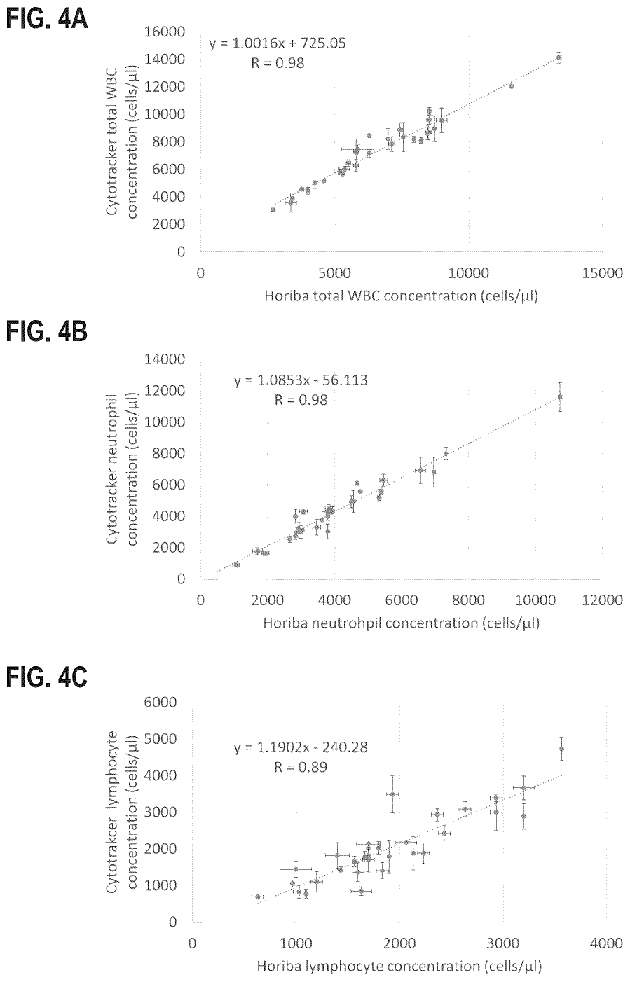

Thirty whole blood samples were purchased from BioIVT. Each blood sample was lysed and measured using three different new cartridges. The coefficient of variation over three devices was calculated. The variation on WBC concentration, neutrophil concentration, and lymphocyte concentration over three new cartridges was within 15%, which indicated that the device variation of the cartridges was small. The correlation analysis was performed between Cytotracker results and Horiba hematology analyzer results on WBC concentration, neutrophil concentration, and lymphocyte concentration. The results are shown in FIGS. 4A, 4B, and 4C. The x-axis is the measured concentration using Horiba hematology analyzer, while the y-axis is CBC results measured using the Cytotracker. The vertical error bar represents the standard deviation of CytoTracker over three devices, and the horizontal error bar reparents the standard deviation of Horiba over three measurements. Within 5 minutes, data showed a correlation coefficient (R) value of 0.98 to the CBC results in terms of total WBC concentration and 0.98 in terms of neutrophil concentration, and 0.89 in terms of lymphocyte concentration. The correlation coefficient (R) value indicated that the CytoTracker results have a good correlation with CBC results provided by the predicate device.

To determine the concentration of a biological entity (e.g., WBC), a fluid velocity of the biological entity is first calculated. The fluid velocity can be determined based on the peak width, which is the transit time of the cells flowing through the electrodes (see Equation 1). Given the fluid velocity and the volume across the electrodes, the flow rate can then be calculated (see Equation 2). Next, the concentration of the biology entity (e.g., WBC in a lysed sample) can be derived by dividing the cell count in unit time by the flow rate (see Equation 3).

$$Transit time = peak width / sampling rate \qquad ( Equation 1 )$$

$$Flow rate = volume above the electrodes / transit time \qquad ( Equation 2 )$$

$$Concentration = count / flow rate \qquad ( Equation 3 )$$

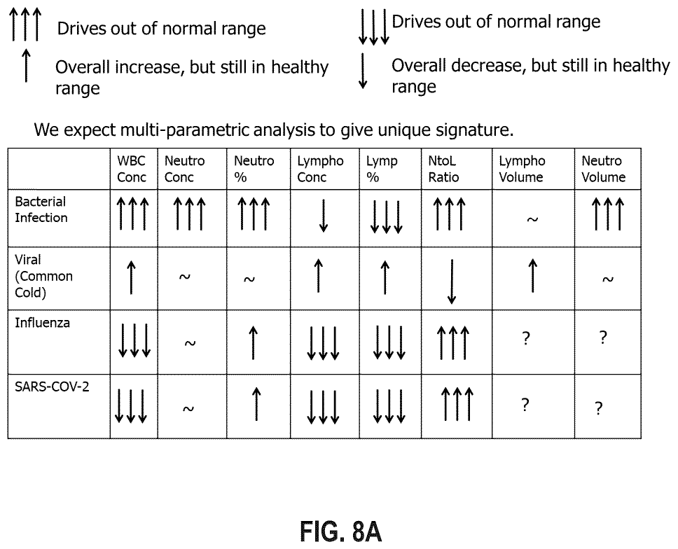

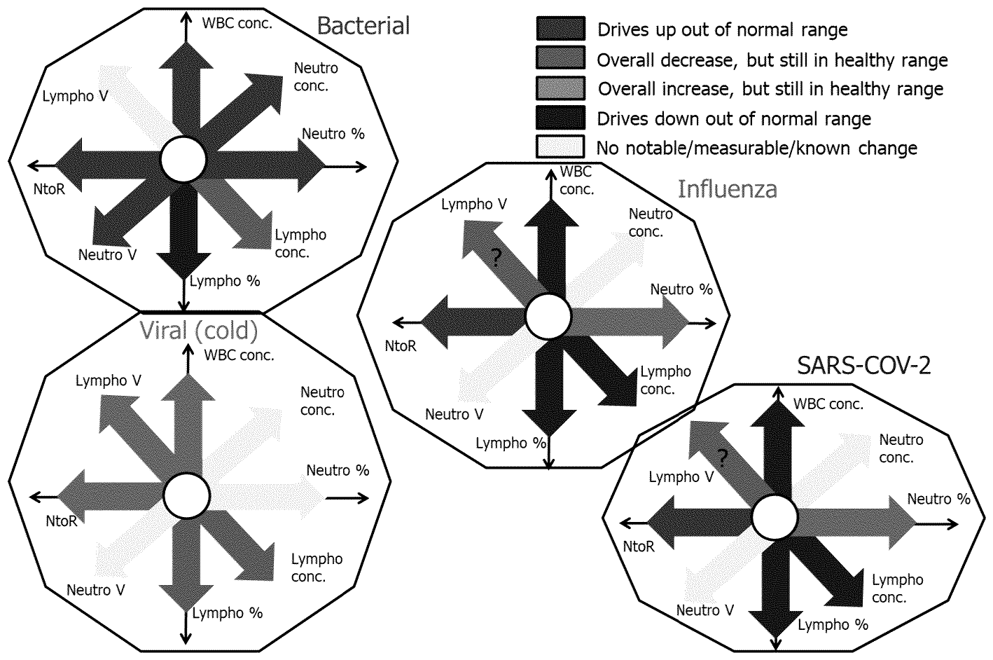

With reference to FIGS. 8A and 8B, white blood cells are the immune system's key players. Neutrophils provide the front line of defense to invasion by bacterial pathogens, whereas lymphocytes do so for viral infections. Generally speaking, in bacterial infections, white blood cell and neutrophil counts increase while the lymphocyte count tends to go down, which is the opposite of what happens in a viral infection. Given the heterogeneity of infections, this trend is not uniform in all cases. The CytoTracker has an AI powered classification engine that takes in these parameters and makes a decision as to whether the infection is viral or bacterial. Machine learning classifiers that can be potentially used include but are not limited to a linear and gaussian support vector machine, neural networks, bagged trees, deep learning, etc. Both supervised and unsupervised classifiers can be used. The features extracted from the microimpedance cytometer include white blood count, neutrophil count, lymphocyte count, neutrophil to lymphocyte ratio, neutrophil percentage, lymphocyte percentage, flag for high or low White blood cell count, flag for high or low neutrophil count, flag for high or low lymphocyte count, electrical impedance peak response at low frequencies (50 kHz to 1 MHZ) and high frequencies (1 MHz to 100 MHz). Monocyte count and monocyte percentage, and high or low level flag can also be added as a feature in other embodiments.

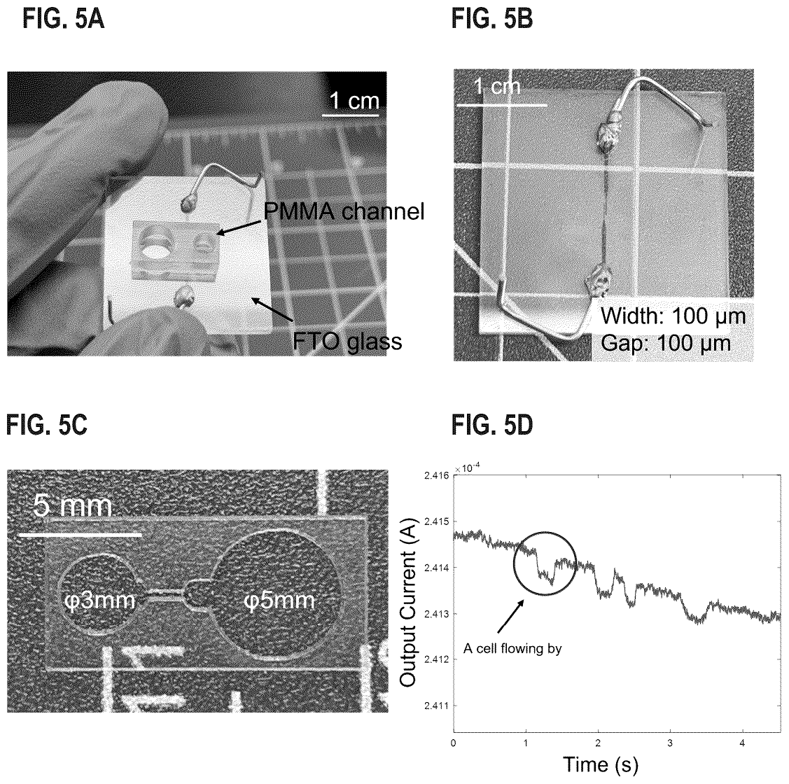

To lower the cost of every single cartridge, the combination of FTO coated glass and CNC fabricated PMMA channel was investigated (FIG. 5). The electrodes are patterned by etching the FTO layer on the glass using a laser. The dimension of the electrodes is 100 μm, and the gap between the two electrodes is 100 μm. PMMA was used as the primary material for the microfluidic channel. It has better hydrophilicity compared with PDMS and is easier to manufacture. The PMMA piece is trimmed using a CNC machine. The sizes of the inlet well and outlet well are 3 cm in diameter and 5 cm in diameter. To integrate the two parts, 3M™ Microfluidic Diagnostic Tape was used. It is double-sided adhesive and hydrophilic. The thickness of the tape is 25 μm. The pattern was designed on the computer and cut the tape using a laser cutter. The power and the speed of the laser beam are optimized to be sufficiently cutting through the tape, while not burn extra materials and causes a wider channel. The detection mechanism of the FTO/PMMA cartridge is similar to the glass/PDMS cartridge. When a cell flows through the sensing region, the current conducting between two electrodes, which is inversely proportional to the electrical impedance, is partially blocked. The current is probed continuously by a lock-in amplifier. To increase the sensitivity of the sensor, the excitation voltage was increased from 1V to 5V. As more current is conducting between electrodes, the perturbation is more obvious to detect. FIG. 5C shows the typical impedance change (output current) when cells were passing by in a 4.5-second time window. Each current drop indicates a cell flowing by. By investigating the amplitude of the current drop, different cell types could be differentiated. The cost of the FTO/PMMA cartridge is significantly decreased, especially for manufacturing. Thus, it can be a good substitution for the glass/PDMS cartridge.

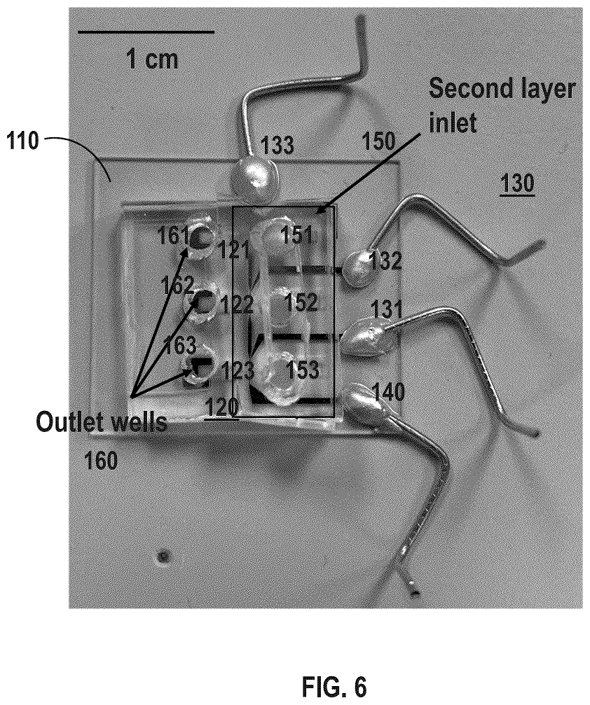

With reference to FIG. 6, there are three microfluidic channels on the single chip. Each channel has a separate inlet well, which is the second layer inlet. To ensure the flows in the three channels are similar, a separate outlet well was added for each channel. The fluid experiences the same capillary force in each channel and avoids interference from the other two to increase the flow rate.

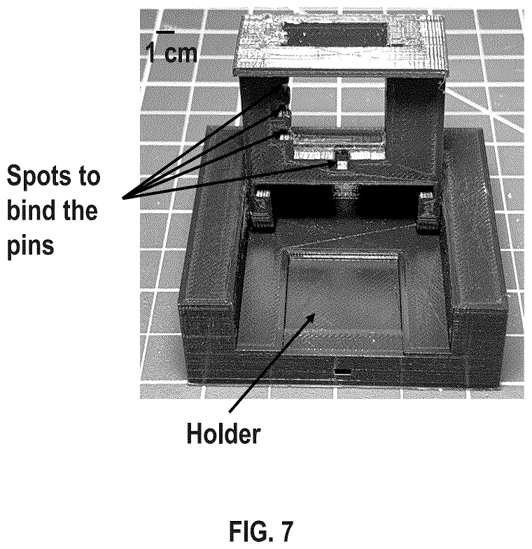

With reference to FIG. 7, an example holder (or package) for the disclosed microfluidic cartridge is provided. As shown, the pins bound to the holder are soldiered with wires or onto a printed circuit board (PCB), which has a connection to the lock-in amplifier.

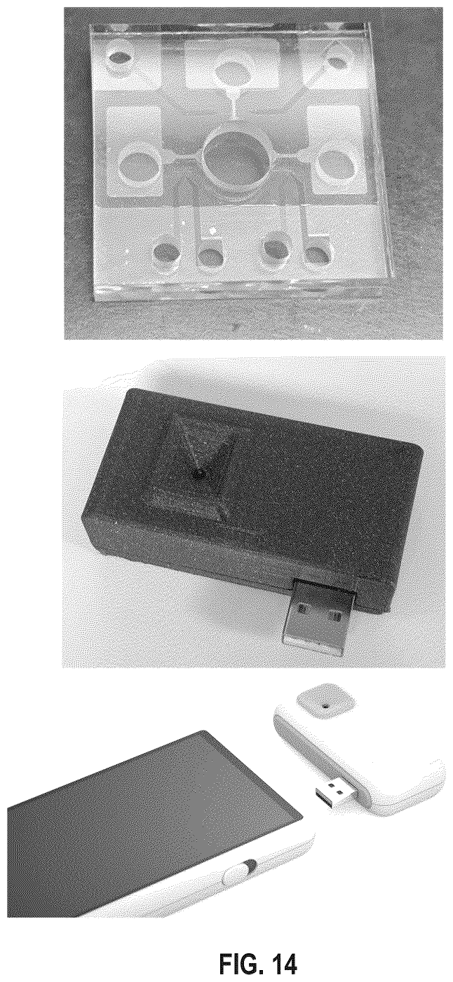

FIG. 14 shows an example design of the disclosed microfluidic system. The microfluidic system may be provided as a test strip and in a plug-n-play or other portable format. It may be powered by, and its signals can be read and analyszed by an extenable (and portable) device.

### Example 2

The rising antimicrobial resistance (AMR) has become a looming threat all over the world. The next pandemic could be the pandemic of AMR and by 2050, 10 million people will die per year. The cause of this pandemic is that antimicrobial resistance is growing, while antimicrobial drug development is slowing. Now more than ever, antibiotic stewardship is of utmost necessity to prevent antimicrobial resistance, while improving patient outcomes. The key driver of AMR is the overuse of antibiotics. The use of antibiotics prompts the selection process that the bacteria with antimicrobial resistance can survive and even multiply. According to the CDC, between 30-50% of antibiotic usage is either unnecessary or inappropriate. 2.5 billion antibiotic prescriptions are made per year globally. The key culprit of antibiotic overuse is the use of antibiotics to treat viral infections that have been mistakenly diagnosed as bacterial infections.

With the help of routine diagnostic tests such as culture tests and polymerase chain reaction (PCR) tests, discriminating between viral and bacterial infections is still challenging. Laboratory culture tests generally take a long incubation time (hours to days) to get results. Besides, contamination happens commonly in the whole process from sample collection to storage and incubation, and thus, leads to false results, unnecessary antibiotic treatment and longer hospital stays. On the other hand, PCR-based tests are more sensitive and reproducible while are less time-consuming. However, these tests are limited to the specific pathogen strain, and it's challenging to develop a reliable assay for emerging and highly variable pathogens. In addition, environmental contamination, most likely carryover contamination, can mislead the results of PCR tests.

A potential approach that could address these challenges relies on monitoring the white blood cell (WBC) count with differentials of the patient. WBCs or leukocytes are critical in the immune system to protect the body against infections and other diseases. WBC counts fluctuate in the immune response to fight infections. It has been widely shown in the literature that high white blood counts, high neutrophil percentages, high neutrophil-to-lymphocyte ratios, and relatively low lymphocyte percentages tend to correlate with bacterial infections. Recent studies investigated WBC count with differentials as a potential marker for infectious diseases. For example, a retrospective and observational study was conducted to explore using the WBC count with differentials in diagnosing bacterial infections in Emergency Department (ED). They demonstrated that neutrophils and total WBC count were the two most useful leukocyte parameters for the diagnosis in the ED. In addition, neutrophil to lymphocyte count ratio (NLCR) has been investigated as an indicator for diagnosis of bacterial infections in many studies. A retrospective study showed that NLCR could be a tool in diagnosing bacterial infection among hospitalized patients with fever. However, most of these studies only investigate a single parameter as the indicator rather than combing multiple parameters as the indicator for the diagnosis.

To explore the usage of the combination of WBC count and the differentials count as the indicator to discriminate bacterial infection from a viral infection, a study was conducted over three sources (Baylor College of Medicine, Robert Wood Johnson Medical School and BioIVT). In addition, a machine learning classifier was implemented to help with the differentiation. The aims of this study were threefold: to verify the capability of CytoTracker to measure the WBC, granulocyte, and lymphocyte concentration correctly; to find the difference in WBC test results between bacterial infection and viral infection; to integrate the findings into a machine learning classifier to differentiate two types of infections.

### Methods

### Study Design and Setting

210 patients were recruited from three different sources: Baylor College of Medicine (n=53), Robert Wood Johnson Medical School (n=99) and BioIVT (n=58). BioIVT is a biological product provider. The patients recruited by BioIVT were outpatient, while the patients enrolled from the other two sites were presented to the emergency department of the hospitals. The venous blood samples were collected from May 2021 to November 2021. The study protocol was approved by Institutional Review Board for Baylor College of Medicine and Affiliated Hospitals (H-49795) and Rutgers University electronic Institutional Review Board (Pro2021001264).

All patients enrolled in the study were adults (>18 years) with suspected/confirmed of viral (COVID positive or other viral infection) and bacterial infections (lower UTI, pneumonia, septicemia, appendicitis, and other relevant bacterial infections, etc). Patients with known white blood cell, neutrophil, and lymphocyte disorders were excluded. Active cancer patients and patients who received chemotherapy in the last 3 months for solid tumor were also excluded. Pregnant patients were not included in the study. Two or more criteria for Systemic Inflammatory Response Syndrome (SIRS 2+) was not an inclusion criterion. Patients received antibiotic treatment were not excluded from analysis.

A venous blood sample was collected from the patient for analyzing using a Horiba ABX Micros 60 Hematology Analyzer (FDA cleared device) and a Cytotracker analyzer. The sample was stored at 4° C. before overnight shipping. The sample tubes were wrapped with absorbent pad and bubble wrap, and placed in a styrofoam box with cold packs during the shipment.

### Clinical Classification and Data Collection

The diagnosis of infection for each patient was determined by typical symptom presentation and/or lab tests. Blood culture tests, urine culture tests, and respiratory culture tests were performed to identify the bacterial infection. Polymerase chain reaction (PCR) tests were used for viral infection detection. Deidentified information was abstracted from the medical chart including CBC values (assessed by the clinical laboratory), diagnostic lab tests, body temperature, date of sample collection, drug treatments (duration, dose), diagnosis for admission, and clinical status. Data from medical chart also included physician's diagnosis (based on lab tests or clinical picture), laboratory test results (hematology, culture results, gram stain), disease course, diagnostic body temperature, and other symptoms.

### CytoTracker Test

Samples were evaluated using an early prototype version of the CytoTracker test (Rizlab Health, Princeton, NJ). First, a ten μl aliquot of EDTA-anticoagulated whole blood sample was treated with the lysing solution and followed by mixing with the quenching solution. Red blood cells were lysed, and the debris was reduced in preparation. Next, three 20 μl aliquots of the lysed sample were transferred to the microfluidic multichannel cartridge. The cartridge was connected to a read-out device for data acquisition and initial processing. Data were collected for five minutes and sent to a local computer. Then, signal processing and data analysis were performed to obtain the cell counts of different types of cells and calculate the concentrations. The test provides total WBC concentration, granulocyte concentration, and lymphocyte concentration.

### Data Analysis and Statistical Analysis

Data analysis and statistical analysis were performed using MATLAB (MathWorks, Natick, MA, USA). The level of significance for all statistical tests was a 2-sided, p-value of 0.05. Student's t-tests were performed to evaluate the difference on total WBC concentration, granulocyte concentration, lymphocyte concentration, granulocyte percentage, lymphocyte percentage, and granulocyte to lymphocyte ratio separately between bacterial infected patient samples and viral infected patient samples. A one-way multivariate analysis of variance (MANOVA) was used to test the difference between two types of infections based on the parameters mentioned earlier. To differentiate bacterial infection from viral infection, a machine learning algorithm was utilized.

### Results

### Patient Characteristics

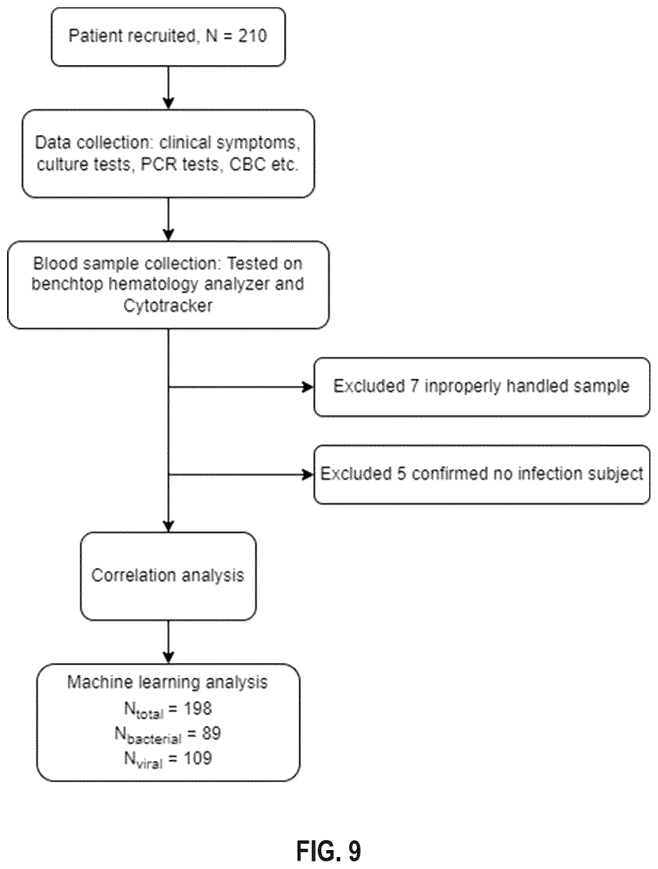

Initially, the study recruited 210 patients with symptoms and collected blood samples (FIG. 9). Seven patients were excluded due to the sample being improperly handled during shipment. The culture and PCR test results indicated bacteria or viruses did not infect five patients, and thus the five were excluded. The final analysis was obtained from 198 patients enrolled over three sources, 49 from BioIVT, 51 from Baylor College of Medicine, and 98 from Robert Wood Johnson Medical School (FIG. 9).

### Assessment of the CytoTracker

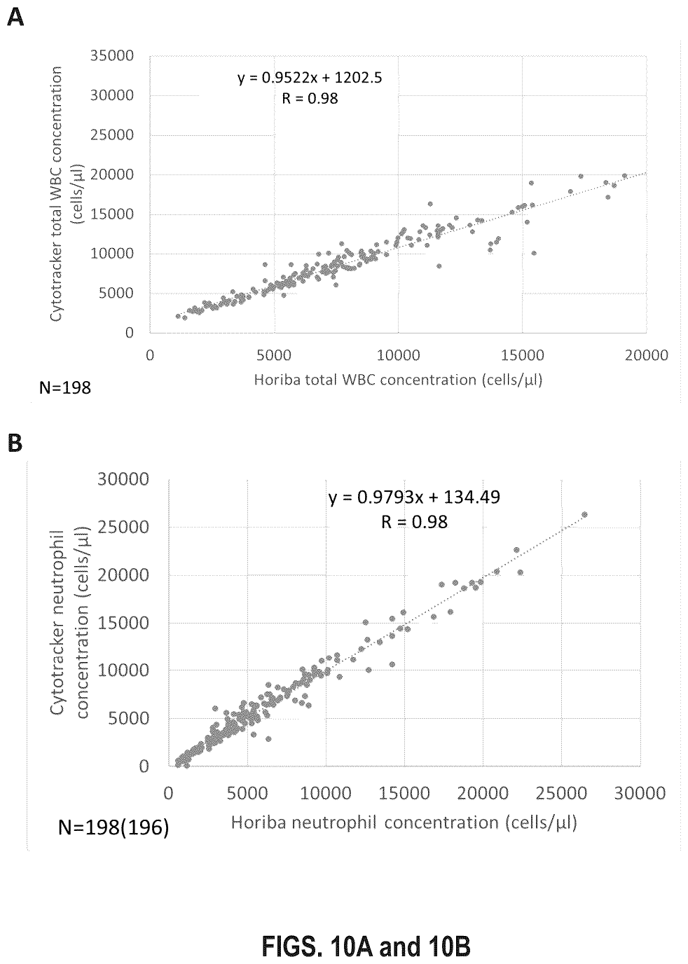

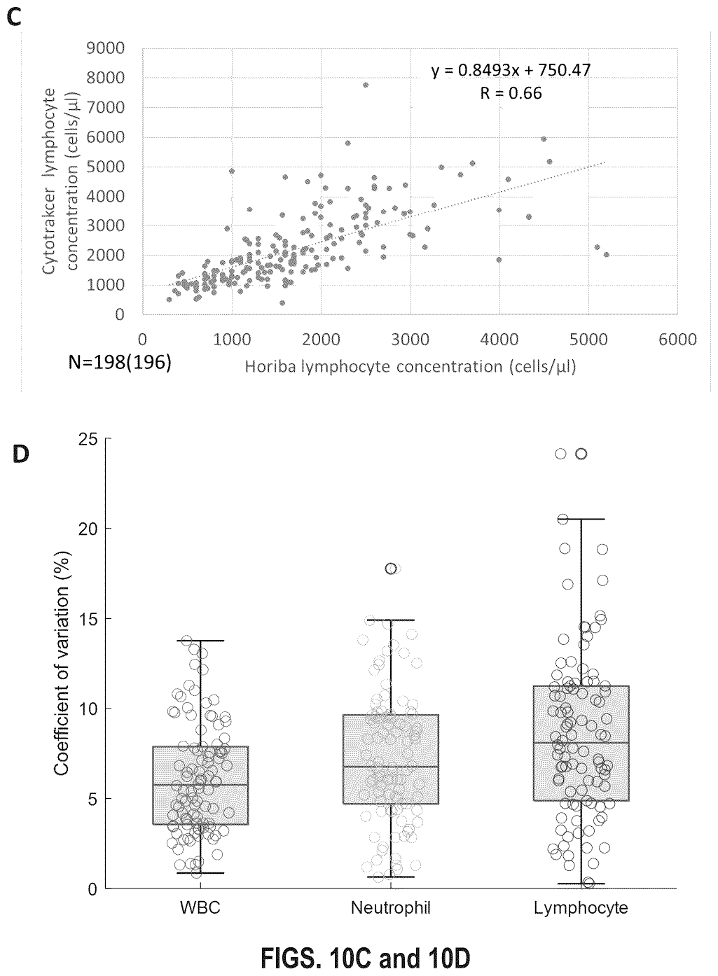

The first goal of this study is to examine whether Cytotracker can accurately estimate the WBC and differentials concentrations, the capability of CytoTracker was assessed in two aspects: correlation with predicate device and the variation from device to device. All samples (n=198) were tested with Horiba ABX micros 60 hematology analyzer to obtain the true value before measuring samples with CytoTracker. A 10 μl aliquot of blood sample was lysed, then pipetted into the Cytotracker cartridge. After 5 minutes measurement, the results were calculated based on captured signals. A correlation analysis was performed on the results of total WBC concentration, granulocyte concentration, and lymphocyte concentration between two devices. The results are shown in FIGS. 10A, 10B, 10C, and 10D. The x-axis is the measured concentration using Horiba hematology analyzer, while the y-axis is CBC results measured using the Cytotracker. Within 5 minutes, data showed a correlation coefficient (R) value of 0.98 to the CBC results in terms of total WBC concentration and 0.99 in terms of granulocyte concentration, and 0.66 in terms of lymphocyte concentration. The correlation coefficient (R) value indicated that the CytoTracker results have a good correlation with CBC results provided by the predicate device.

To evaluate the device-to-device variation of the CytoTracker measurements, blood sample was lysed three times and measured using three different new cartridges. The coefficient of variation (CV) was calculated over three measurements. FIG. 10D shows the CV of WBC concentration, granulocyte concentration, and lymphocyte concentration. The variation on WBC concentration over three new cartridges was within 15%, which indicated that the device variation of the cartridges was not large.

### Blood Count Difference Between Bacterial Infection and Viral Infection

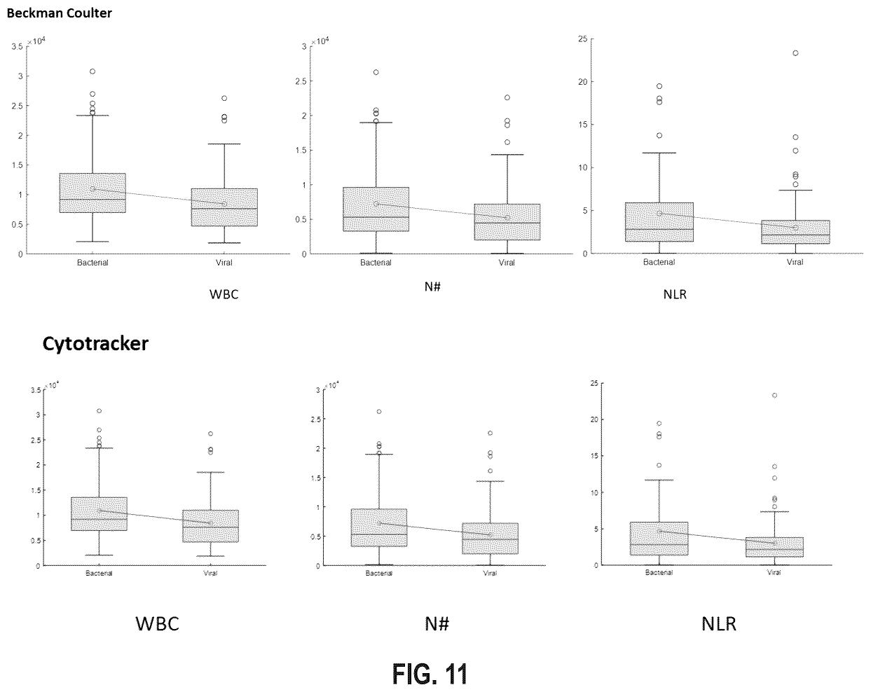

The difference on total WBC concentration, granulocyte concentration, lymphocyte concentration, granulocyte percentage, lymphocyte percentage, and granulocyte to lymphocyte ratio was investigated between bacterial infected patient samples and viral infected patient samples. FIG. 11 demonstrates the box plot of each parameter between two types of infections based on the CytoTracker results. The blue circles represents for outliers and the red circles represents for the average value. Student's t-test was also performed to evaluate the statistical significance. The results showed that the total WBC concentration, granulocyte concentration, and granulocyte to lymphocyte ratio of bacterial infected patients are higher than those of viral infected patients (p<0.05). Similar 20) results were observed using Beckman Coulter results (SI).

Next, a one-way multivariate analysis of variance (MANOVA) was conducted to test the hypothesis that there would be a mean difference between two type of infections. A statistically significant MANOVA effect was obtained, p<0.05. It indicates that the CBC results of bacterial infected patient is different from that of viral infected patient.

### Feature Performance and Machine Learning Analysis

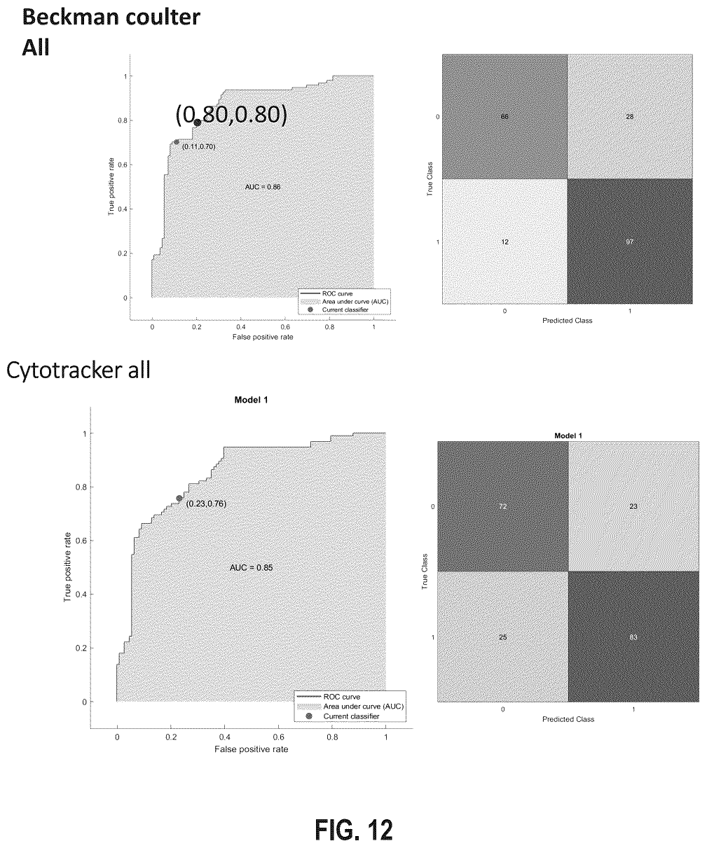

To examine whether the blood count difference between two types of infections can be used as features for differentiation, a machine learning analysis was conducted. The performance of classifiers was compared using different combinations of parameters, including total WBC concentration, granulocyte concentration, lymphocyte concentration, granulocyte percentage, lymphocyte percentage, granulocyte to lymphocyte ratio, and the flags assigned for each. If the above concentration/percentage is below, within, or above the normal range, the flag were assign as −1, 0, or 1 accordingly. Because the normal range of the granulocyte to lymphocyte ratio is not well established, the flag was not used for the granulocyte to lymphocyte ratio as a feature for the analysis. FIG. 12 shows the AUC results of each classifier.

What is claimed is:

1. A microfluidic system for impedance-based detection of a biological entity in a sample, comprising:
a substrate;
two or more microfluidic flow channels positioned on the substrate, wherein the microfluidic flow channels are configured to conduct passage of the biological entity;
at least one inlet formed on the substrate, wherein the at least one inlet is configured to receive the sample and in fluid communication with the microfluidic flow channels;
at least one outlet formed on the substrate, wherein the at least one outlet is in fluid communication with the microfluidic flow channels and configured to receive the sample after the sample flows through the microfluidic channels; and
an impedance circuit disposed on the substrate, comprising two or more excitation electrodes and a common electrode,
wherein each of the excitation electrodes is respectively coupled to each of the microfluidic flow channels and configured to be electrically connected to a signal generator, wherein the excitation electrodes are configured to receive and electrically communicate an excitation signal applied by the signal generator to each of the microfluidic flow channels, wherein the excitation signal generates an electric field between each of the excitation electrodes and the common electrode, and
wherein the common electrode is coupled to all of the microfluidic flow channels and configured to be electrically connected to an impedance analyzer, wherein the common electrode is configured to electrically communicate an output signal to the impedance analyzer, and wherein the output signal correlates to an impedance variation caused by displacement of the biological entity within each of the microfluidic flow channels.

2. The system of claim 1, wherein the microfluidic flow channels are configured to conduct passage of the biological entity therethrough simultaneously.

3. The system of any one of the preceding claims, wherein the microfluidic flow channels comprise three microfluidic flow channels.

4. The system of any one of the preceding claims, wherein the microfluidic flow channels are formed on or affixed to the substrate.

5. The system of any one of the preceding claims, wherein the at least one inlet comprises three inlets and the at least one outlet comprises three outlets.

6. The system of any one of claims 1-4, wherein the at least one inlet comprises one inlet and the at least one outlet comprises three outlets.

7. The system of any one of the preceding claims, wherein the microfluidic flow channels are of the same dimension.

8. The system of any one of the preceding claims, wherein the microfluidic flow channels comprise a microfluidic flow channel having a width of from about 70 to about 90 micrometers and a height of from about 18 to about 22 micrometers.

9. The system of claim 8, wherein the microfluidic flow channel has a width of about 80 micrometers and a height of about 20 micrometers.

10. The system of any one of the preceding claims, wherein the microfluidic flow channels have a circular, oval, or polygonal cross-section.

11. The system of any one of the preceding claims, wherein the excitation electrodes or the common electrode have a width of from about 10 to about 50 micrometers.

12. The system of claim 11, wherein the excitation electrodes or the common electrode have a width of about 25 micrometers.

13. The system of any one of the preceding claims, wherein the excitation electrodes are spatially disposed on the substrate with a gap between two electrodes of from about 10 to about 50 micrometers.

14. The system of claim 13, wherein the gap between two electrodes is about 20 micrometers.

15. The system of any one of the preceding claims, wherein the inlet or the outlet has a diameter of from about 2 to about 8 centimeters.

16. The system of claim 15, wherein the inlet has a diameter of about 3 centimeters, and the outlet has a diameter of about 5 centimeters.

17. The system of any one of the preceding claims, wherein the signal generator comprises a function generator.

18. The system of any one of the preceding claims, wherein the impedance analyzer comprises a lock-in amplifier.

19. The system of any one of the preceding claims, wherein the output signal is proportional to the impedance variation of the biological entity within the each of the microfluidic flow channels.

20. The system of any one of the preceding claims, wherein the excitation signal has a frequency of from about 100 kHz to about 20 MHz.

21. The system of any one of the preceding claims, wherein the signal generator applies a different frequency of the excitation signal to each of the excitation electrodes.

22. The system of claim 21, wherein the microfluidic flow channels comprise three microfluidic flow channels, and the signal generator applies three different frequencies of the excitation signal respectively to the three microfluidic channels.

23. The system of claim 21, wherein the three different frequencies are about 490 kHz, about 500 kHz, and about 510 kHz, respectively.

24. The system of any one of the preceding claims, wherein the excitation signal comprises sinusoidal excitation signals.

25. The system of any one of the preceding claims, wherein the impedance analyzer demodulates impedance responses of the microfluidic flow channels from the output signal received from the common electrode.

26. The system of any one of the preceding claims, wherein the substrate is formed of a polymer material.

27. The system of claim 26, wherein the substrate is formed of polymethyl methacrylate (PMMA) or fluorine-doped tin oxide (FTO)/PMMA.

28. The system of any one of the preceding claims, wherein the biological entity comprises any one of red blood cell, white blood cell, platelet, hematocrit, hemoglobin, neutrophil, lymphocyte, microbial, and a combination thereof.

29. The system of any one of the preceding claims, comprising two layers of the substrate, wherein the two layers of the substrate are patterned with metal and affixed to each other by adhesive, wherein space generated by the adhesive forms the microfluidic flow channels.

30. The system of claim 29, wherein the microfluidic flow channels of a size of about 25 micrometers.

31. The system of claim 29, wherein the two layers of the substrate comprise a glass layer.

32. The system of claim 29, wherein the two layers of the substrate are patterned by laser patterning.

33. The system of claim 29, wherein the metal comprises indium tin oxide, fluorine tin oxide, gold, aluminum, platinum, graphene, graphene oxide, reduced graphene oxide, molebdium disulfide, silver, silver chloride, copper, graphite, titanium, steel, brass, or a combination thereof.

34. The system of claim 29, wherein the adhesive comprises pressure sensitive adhesive.

35. A kit comprising the system of any one of the preceding claims.

36. A method for identifying or counting a biological entity in a sample, comprising:
providing the microfluidic system of any one of claims 1-34;
applying the sample to the at least one inlet;
applying an excitation signal to the excitation electrodes by the signal generator for a period of time;
receiving an output signal communicated from the common electrode;
determining an impedance variation caused by displacement of the biological entity within the microfluidic flow channels; and
determining a type or a number of the biological entity in the sample based on the impedance variation.

37. A method of diagnosing a disease or disorder in a subject, comprising:
providing the microfluidic system of any one of claims 1-34;
applying the sample to the at least one inlet;
applying an excitation signal to the excitation electrodes by the signal generator for a period of time;
receiving an output signal communicated from the common electrode;
determining an impedance variation caused by displacement of the biological entity within the microfluidic flow channels;
determining a number of the biological entity in the sample based on the impedance variation; and
determining that the subject has the disease or disorder if a difference between the number of the biological entity and a control level is greater than a threshold value.

38. A method of monitoring progression of a disease or disorder in a subject, comprising:
providing the microfluidic system of any one of claims 1-34;
applying the sample to the at least one inlet;
applying an excitation signal to the excitation electrodes by the signal generator for a period of time;
receiving an output signal communicated from the common electrode;
determining an impedance variation caused by displacement of the biological entity within the microfluidic flow channels;
determining a number of the biological entity in the sample based on the impedance variation and determining if the number of the biological entity is elevated or decreased as compared to a second control level; and
determining that (a) the subject has progression of the disease or disorder if the number of the biological entity is elevated as compared to the second control level; and (b) the subject has regression of the disease or disorder if the number of the biological entity is decreased as compared to the second control level.

39. The method of any one of claims 36-38, wherein the excitation signal has a frequency of from about 100 kHz to about 20 MHz.

40. The method of any one of claims 36-39, comprising applying by the signal generator a different frequency of the excitation signal to each of the excitation electrodes.

41. The method of claim 40, wherein the microfluidic flow channels comprise three microfluidic flow channels; and the method comprises applying by the signal generator three different frequencies of the excitation signal respectively to the three microfluidic channels.

42. The method of claim 41, wherein the three different frequencies are about 490 kHz, about 500 kHz, and about 510 kHz, respectively.

43. The method of any one of claims 36-42, wherein the excitation signal comprises sinusoidal excitation signals.

44. The method of any one of claims 36-43, comprising demodulating by the impedance analyzer impedance responses of the microfluidic flow channels from the output signal received from the common electrode.

45. The method of any one of claims 36-44, comprising applying a wavelet filter to the output signal.

46. The method of any one of claims 36-45, further comprising applying a Hampel filter to the output signal.

47. The method of any one of claims 36-46, wherein the biological entity comprises a bacterium, a virus, a protein, a microparticle, a nanoparticle, a nucleic acid, a biomarker, or a bead with a biological material attached thereto.

48. The method of any one of claims 36-46, wherein the biological entity comprises any one of red blood cell, white blood cell, platelet, hematocrit, hemoglobin, neutrophil, lymphocyte, microbial, and a combination thereof.

49. The method of claim 48, comprising determining a number, a concentration, or a percentage of one or more of white blood cells, lymphocytes, and neutrophils in the sample.

50. The method of any one of claims 36-49, comprising determining a neutrophil:lymphocyte ratio.

51. The method of claim 50, comprising identifying a disease or disorder or monitoring progression of the disease or disorder by comparing the neutrophil:lymphocyte ratio to a control ratio.

52. The method of any one of claims 37-51, comprising identifying a disease or disorder or monitoring progression of the disease or disorder based on one or more characteristics selected from white blood cell counts, concentration of neutrophils, percentage of neutrophils, volume of neutrophils, concentration of lymphocytes, percentage of lymphocytes, volume of neutrophils, volume of lymphocytes, neutrophil to lymphocyte ratio, flagging high or low WBC levels, neutrophil levels, or lymphocytes, volume distribution skew, conductivity and electrical scattering properties of cells, membrane capacitance and conductivity, cytoplasm electrical properties, and others.

53. The method of claim 52, wherein identifying a disease or disorder or monitoring progression of the disease or disorder is performed by a machine learning module.

54. The method of any one of claims 37-53, wherein the disease or disorder is a bacterial or viral infection.

55. The method of claim 54, wherein the disease or disorder comprises influenza or SARS-CoV-2.

56. The method of any one of claims 36-55, wherein the sample comprises a bodily fluid.

57. The method of claim 56, wherein the bodily fluid comprises blood.

58. The method of any one of claims 36-57, further comprising contacting the sample with a lysis reagent for a period of time.
